(19) United States
(12) Patent Application Publication
(10) Pub. No.: US 2012/0225094 A1
(43) Pub. Date: Sep. 6, 2012

(54) Treatment of Sleep Disordered Breathing with Neurotoxin

(76) Inventor: Ira Sanders, North Bergen, NJ (US)

(21) Appl. No.: 13/508,930

(22) PCT Filed: Nov. 9, 2010
(86) PCT No.: PCT/US10/56086

§ 371 Date: May 9, 2012

Related U.S. Application Data
(60) Provisional application No. 61/259,629, filed on Nov. 9, 2009.

(51) Int. Cl. A61K 39/08; A61M 5/00; A61P 11/00; A61M 5/307

(52) U.S. Cl. 424/239.1; 604/500; 604/506

(57) ABSTRACT

Disclosed herein are compositions of neurotoxins and methods of their use for the treatment of sleep disordered breathing. In one embodiment of the present invention, a method of treating sleep breathing disorders comprising administering a therapeutically effective amount of Clostridia neurotoxin (CnT) or light chain thereof to a mammal in need thereof is disclosed.

### CLAIM OF PRIORITY

This application claims priority to U.S. Provisional Application No. 61/259,629 titled “Treatment of Sleep Disordered Breathing with Neurotoxin,” filed on Nov. 9, 2009, the disclosure of which is incorporated by reference herein in its entirety.

### BACKGROUND OF THE INVENTION

### Obstructive Sleep Apnea

Upper airway (UA) patency is dependent on the activity of pharyngeal dilator muscles. Humans are unique because their upper airway has a curved shape, an anatomical change that is related to the evolution of human speech. As a result, the UA of humans is more flexible than other species and is more prone to collapse under negative pressure. While awake, humans have continuous tone in their upper airway muscles that keeps the passageway open. However, during sleep the tone and reflex activity of UA muscles decreases, and in susceptible individuals, this leads to pharyngeal narrowing which can interfere with breathing.

Sleep disordered breathing (SDB) is the medical name that encompasses a wide variety of breathing problems during sleep. SDB represents a spectrum of disorders varying in severity and including snoring, upper airway resistance syndrome (UARS), hypopnea (<50% airflow) and apnea (no airflow). SDB becomes a formal medical condition, called obstructive sleep apnea (OSA), when the patient experiences more than 5 episodes of either hypopnea or apnea lasting more than 10 seconds during each hour of sleep. Snoring is included within the above description of sleep disordered breathing because it is a common symptom of SBD. Although it is a common partner complaint, snoring is not a medical condition. Snoring is merely the sound of the vibrations of upper airway tissues. Many individuals have snoring without accompanying SDB.

OSA is diagnosed by an overnight polysomnography (PSG) recording that measures multiple respiratory, cardiovascular, and central nervous system parameters. The severity of OSA is measured by the number of apneas and hypopneas during each hour of sleep and is expressed as the apnea-hypopnea index (AHI), also called the respiratory disturbance index (RIM). The American Academy of Sleep Medicine defines various levels of severity for OSA: mild (AHI 5-15); moderate (AHI >15-30); and severe (AHI>30).

### Pathophysiology of Obstructive Sleep Apnea

Sleep has four non-rapid eye movement (NREM) stages and a fifth stage of rapid eye movement (REM). These stages are marked by progressively greater muscle relaxation with UA muscle activity reaching its minimum during REM. The relaxation of UA muscles narrows the UA and decreases airflow thereby causing hypopneas and apneas. These episodes of decreased airflow often cause some degree of arousal during sleep. Although the patient does not awaken to full consciousness, the sleep pattern is disturbed and the patient shows signs of sleepiness and fatigue during waking hours. Even greater than normal inspiratory effort that does not meet the criteria of apnea or hypopnea can cause sleep disturbance. This condition is called upper airway resistance syndrome (LIARS), a form of SDB that doesn't display medically significant hypopnea yet results in hypersomnolence.

The upper airway (UA) refers to the air filled spaces between the nose and mouth and the larynx. The shape and flexibility of the UA, combined with risk factors for OSA, can lead to UA collapse (although other mechanisms may sometimes contribute). The retropalatal area is more susceptible to collapse because it is the narrowest area of the UA and contains two overlapping flexible structures, the soft palate and tongue. Specifically, the key structure of the retropalatal area involved in OSA is the curved part of the tongue base. This structure is highly compliant when relaxed and any reduction in UA pressure affects this part of the UA the most.

### Prevalence and Importance of OSA

The prevalence of OSA ranges from 4-20% of the population. The frequently cited Wisconsin Sleep Cohort Study found that in people age 30-60 years, 28% of men and 9% of women had an AHI greater than 5. Almost all studies suggest that a majority of OSA patients, perhaps as many as 80%, are undiagnosed. Therefore, recent increases in the incidence of OSA largely reflect the diagnosis of existing OSA patients.

The major risk factors for OSA are obesity, sex (male-to-female ratio is about 3:1), and age (increased incidence in older population). With the growing epidemic of obesity in an aging population, it is likely that the incidence of OSA will rapidly increase.

Sleepiness (hypersomnolence) is the most noticeable symptom of OSA, and episodes of decreased airflow often cause some degree of arousal during sleep. Although the patient does not awaken to full consciousness, the normal sleep pattern is disturbed and the patient shows signs of sleepiness and fatigue during waking hours. This is believed to be a major cause of industrial and traffic accidents. The National Transportation Safety Board estimates that each year 100,000 traffic accidents resulting in 1500 fatalities are directly attributable to OSA.

More importantly, OSA can lead to debilitating medical disorders and even death. OSA is correlated with myocardial infarctions, cerebrovascular accidents, and chronic hypertension. Epidemiologic studies show that sleep apnea increases risks for cardiovascular disease independent of demographic characteristics (i.e., age, sex, and race) or cardiovascular risk markers (i.e., smoking, alcohol, obesity, diabetes, dyslipidemia, atrial fibrillation, and hypertension). Patients with severe OSA have been found to have a lower 10-year survival rate compared to healthy subjects. Effective treatment of OSA significantly improves cardiovascular outcome by reducing pulmonary and systemic hypertension, reducing arrhythmias and reducing fatal and non-fatal myocardial infarction and stroke. OSA treatment has been shown to reduce blood pressure by as much as 10mm Hg, which in turn reduces coronary heart disease event risk by 37% and stroke risk by 56%. Current evidence also points to a reduction in daytime sleepiness and motor vehicular accidents. Effective OSA treatment also reduces mortality and improves survival.

Gone untreated, OSA is a major contributor to the incidence of hypersomnolence, depression, acid reflux, hypertension, heart failure, atrial fibrillation, myocardial infarction, cerebral vascular accident, metabolic syndrome, traffic accidents, and industrial accidents.

### Current Treatment of OSA

OSA is clearly a major public health problem. Current therapies, which range from behavioral therapy to oral/dental devices to surgical intervention, have been inadequate.

Continuous positive airway pressure (CPAP) devices have improved substantially and remain an effective form of therapy for adult SDB. However, they are cumbersome and have achieved only moderate acceptance by patients. Other approaches, such as oral appliances and upper airway surgery, have relatively limited success rates for more than mild to moderate SBD. Therefore, current forms of therapy need to be improved, and novel therapies need to be developed.

(1) Non-Surgical OSA Treatments

Continuous Positive Airway Pressure

The standard treatment for OSA in most countries is continuous positive airway pressure (CPAP). Continuous positive airway pressure (CPAP) is the mainstay of OSA treatment and is used by approximately 3 million patients each year in the United States. CPAP requires pressurized air to be pumped through the nose every night while sleeping to act as a pneumatic stent for the airway. In most patients it is effective in normalizing AHI and reverse the sleepiness associated with OSA.

Although effective, CPAP is perceived as uncomfortable by patients, and disruptive to the spouse, which often leads to poor compliance with therapy. An estimated 50-80% of patients either refuse or are not compliant with CPAP therapy and risk associated medical consequences.

Oral Appliances

Most oral appliances (OA) are of the mandibular advancement type. The patient's teeth are anchored to the device and the mandible is advanced anteriorly relative to the maxilla. As the tongue is coupled to the mandible it is also moved anteriorly, which increases the upper airway diameter. In addition the lateral pharyngeal walls are stretched and tightened, also adding to the pharyngeal airspace.

Oral appliances show improvement of symptoms in OSA, particularly in patients with mild OSA. Well controlled crossover studies comparing OA to a sham control show improvement in sleepiness and a 5 point improvement in AHI scores but no change in oxygen desaturation. However, a majority of the trials have studied only mild to moderate sleep apneics.

(2) Surgical Procedures for OSA

Surgical procedures are used to treat a small proportion of OSA patients treat. Compared to CPAP, all surgical procedures have less efficacy and much higher risk. Surgical procedures include soft palate procedures (e.g. palatal stiffening and uvulopalatopharyngoplasty), tongue volume reduction procedures (e.g. midline glossectomy and radiofrequency ablation), airway expansion procedures (e.g. genioglossus advancement, hyoid myotomy suspension, and bi-maxillary advancement), and airway bypass (e.g. tracheotomy).

### SUMMARY OF THE INVENTION

It is an object of this invention to provide a method of treating sleep breathing disorders comprising administering a therapeutically effective amount of clostridia neurotoxin (CnT) or light chain thereof to a mammal in need thereof.

In certain embodiments, the CnT or light chain thereof is administered to the mammal's nose, nasal cavity, sinuses, oral cavity, pharynx, larynx, trachea or bronchi.

In further embodiments, the sleep breathing disorder is selected from the group consisting of upper airway resistance syndrome, hypopnea, central sleep apnea, and obstructive sleep apnea.

In some embodiments, the CnT or light chain thereof is administered at a dosage of between about 0.01 to 10,000 units. In other embodiments, the CnT or light chain thereof is administered at a dosage of between about 0.1 to 1,000 units. In further embodiments, the CnT or light chain thereof is administered at a dosage of between about 1 to 100 units.

In some embodiments, the CnT or light chain thereof comprises at least one of the botulinum toxin serotypes A, B, C, D, E, F or G. In other embodiments, the CnT or light chain thereof comprises tetanus toxin. In certain embodiments, the CnT or light chain thereof is modified by additions or substitutions of at least 1 amino acid.

In some embodiments, the CnT or light chain thereof is administered topically. In other embodiments, the CnT or light chain thereof is administered by injection. In further embodiments, the CnT or light chain thereof is administered by jet injection or needle injection. In other embodiments, the CnT or light chain thereof is administered by aerosolized spray.

In some embodiments, the CnT or light chain thereof is administered using a sustained release delivery method. In certain embodiments, the sustained release delivery method comprises injecting the mammal with a depot injection, administering the CnT or light chain thereof topically, or administering the CnT or light chain thereof in a bioresorbable carrier.

In certain embodiments, the CnT or light chain thereof is applied to respiratory or oral mucosa. In other embodiments, the CnT or light chain thereof is administered across respiratory or oral mucosa. In further embodiments, the CnT or light chain thereof is administered to the sphenopalatine ganglia.

In some embodiments, the light chains of CnT are administered to the mammal.

It is a further object of the invention to provide a method of treating the symptoms of sleep breathing disorders comprising administering a therapeutically effective amount of clostridia neurotoxin (CnT) or light chain thereof to a mammal in need thereof. In some embodiments, the symptom is snoring.

The airway is divided into upper airway (UA) and the lower airway (LA). The UA begins at the nostrils (skin and mucosa), then includes the nasal cavity and the paranasal sinuses (maxillary, frontal, ethmoid and sphenoid), the pharynx (naso-, velo-, oro-, and hypopharynx) and the larynx at the level of the vocal cords. The oral cavity extends from the lips to the anterior margins of the pharynx. The LA includes the larynx below the vocal folds, trachea, the bronchi (main bronchi to terminal bronchioles), and the alveoli. For the purpose of this disclosure, that part of the larynx above the vocal folds will be considered part of the UA, while that part below the vocal folds will be considered LA.

### DETAILED DESCRIPTION OF THE INVENTION

Unexpectedly, it has been found that application of clostridia neurotoxins (CnT) (botulinum and tetanus toxins) to the oral and respiratory mucosa or surrounding muscular or neural structures can improve sleep disordered breathing. Notably, the dosing of these toxins need not cause muscle weakness. Without being bound by a particular theory, it appears that the CnT affects the reflexes that maintain airway dilation thereby opposing the decreased upper airway muscle tone during sleep. This may be by a direct effect on peripheral sensory structures in the airway mucosa, or by a central effect after retrograde transport of the toxin. Sensory elements are present in the mucosa and submucosa. Particularly high concentrations of mucosal receptors are found in the anterior nasal cavity, posterior nasal cavity and nasopharynx, and the mucosa of the epiglottis, however, sensory elements are found throughout the respiratory and gastrointestinal tract. Some sensory elements are found beneath the mucosa and even in the connective tissue surrounding airway structures such as the sensory neural plexus present between the trachea and esophagus and throughout the lung. Finally, neural ganglia (e.g. Sphenopalatine ganglion) are structures where peripheral neurons are concentrated.

Clostridia neurotoxins (CnT) are defined as botulinum serotypes A, B, C, D, E, F, G and tetanus toxin. CnT also encompasses all modified or substituted versions of these toxins that have the same blocking effect on SNARE proteins. These include any substitution or modification of at least 1 amino acid of a naturally produced toxin. Also included are toxins with removal or substitution of the binding domain and/or translocation domain. Also included are methods of drug delivery including liposomes, protein transduction domains, cationic proteins, acidic solutions and numerous other methods known in the art. Further included are the light chains of these toxins if delivered intracellularly by liposomes, protein transduction proteins, cationic proteins, iontophoresis or other methods known in the art.

Doses of CnT described in the examples are those using botulinum toxin A (Botox®) manufactured by Allergan Inc. (Irvine, Calif.) except where indicated. The unit measure of botulinum toxin potency is the amount that kills 50% of mice when injected into their peritoneum. Although the clinical potency of units from different botulinum toxin products would be expected to be the same, it is well known in the art that their potency differs when injected into humans. The biological equivalence ratios to Botox® are known for current commercial products and can be determined without undue experimentation. Dysport from Ipsen LTD (Bath, England) has ⅓^{rd} the bioequivalence per unit than Botox®. Myobloc (Botulinum toxin type B), Solstice Neuroscience, (Malvern, Pa.) has 1/40^{th} the bioequivalence of Botox®.

CnT is usually injected into small areas of approximately 1 cm², and treatment of larger areas requires multiple injections. The doses given here and in the examples refer to the range needed for an entire treatment. Doses can range, for example and without limitation, from about 0.01 to about 10,000 units, preferably from about 0.1 to about 1,000 units or from about 1 to about 100 units.

Major variations in dose can result from the topical use of botulinum toxin, as a percentage of toxin does not fully penetrate mucosa or skin, and often most of the toxin is wiped away after application, and penetration of actual toxin can be as low as 1%. The amount of toxin referred to in the above dose ranges therefore refers to the actual toxin penetrating within the body and not the total dose applied on the surface. This actual dose is known for topical medications as it always studied and quantified during the FDA approval process.

Doses of, for example and without limitation, about 0.01 to about 10,000 units per square cm can be administered using controlled release methods, such as delayed release, sustained release, or delayed sustained release. Sustained release methods can include, without limitation, slow releasing depot injections, topical preparations, or bioresorbable carriers (e.g. poloxymer), whereby the release of the toxin is delayed or is released over an extended period of time, which, depending on the mode of sustained release technology used, can range from, for example, 1 second, 1 minute, 1 day, 1 month, 3 months, 6 months, etc.

CnT can be applied topically, by injection (including, without limitation, pressure jet injection or needle injection), by aerosolized spray, in a bioresorbable carrier, or by other methods known in the art.

### EXAMPLES

### Example 1

A 50 year old man is diagnosed with mild sleep apnea as reflected by an AHI of 10.

1000 units of CnT in 1 cc of a poloxymer carrier are injected through the sinus opening (ostia) into the left maxillary sinus. The poloxymer solidifies in the maxillary cavity and dissolves over 4 days. Follow-up sleep studies at 1 month show improvement of the AHI to 4, essentially curing the sleep apnea.

Alternatively, both maxillary sinuses can be injected with a dose of 500 units in 0.5 cc of poloxymer carrier.

This example illustrates the method of treating sleep apnea by application of CnT to a sinus cavity. The example also illustrates the use of carriers that provide sustained release of CnT.

### Example 2

A 25 year old male with daytime sleepiness undergoes sleep testing and is found to have an AHI of 4. He is diagnosed with UARS. To treat this condition an ENT doctor anesthetizes the oral cavity. Then, using a laryngeal mirror and curved laryngeal instruments, he injects 50 units of CnT in 0.5 cc of normal saline into the mucosa of the epiglottis. A visible bleb is seen on the lingual side of the epiglottis. After observing the patient for complications he is sent home. In 2 weeks the patient notices a marked improvement in his daytime sleepiness reflected by an Epworth Sleepiness Scale (ESS) rating of 9.

This example illustrates the submucosal injection of CnT to deep pressure receptors located in the epiglottic cartilage.

### Example 3

A 60 year old male is diagnosed with moderate OSA with an AHI of 25. His physician treats the patient by injecting 50 units into the mucosa of the nasopharynx. First the physician decongests the nose with a 1% spray of neosynephrine. Then the nasal mucosa in anesthetized by a 1% lidocaine spray. The physician then introduces a 2.7 mm rigid 0 degree endoscope through the nostril to the back of the nasal cavity. 25 units of CnT are then injected into the posterior end of each inferior turbinate. The patient is observed for complications and then is sent home. Repeat PSG at 1 month shows an improvement in AHI to 14.

### Example 4

Alternatively the same patient described in Example 3 may be treated with topical CnT in dissolvable cellulose (Surgicel, J&J, Somerset, N.J.). 100 units in normal saline are absorbed onto a 1 cm sheet of cellulose (range of possible sizes 1-20 cm). After anesthetizing and decongesting the nasal cavity the cellulose sheeting is draped onto mucosa of the anterior nares, nasal cavity, or nasopharynx. The CnT would be allowed to absorb onto mucosa for 24 hours and the remaining sheeting, if still present, would be expelled.

### Example 5

Alternatively the same patient described in example 3 is treated by administration of 25 units of CnT to his Sphenopalatine ganglion bilaterally by injection through the Sphenopalatine canal. Alternatively the CnT can be administered to the Sphenopalatine ganglion by topically applying 50 units of CnT to the posterior nasal wall overlying the ganglion on one or both sides of the nasal cavity.

### Example 6

Alternatively, the same patient described in example 3 is treated by inhaling a solution of 10 units of CnT in aerosolized normal saline. The aerosolized particles are sized to deposit in the trachea and upper bronchi and not to reach the alveoli where they may be systemically absorbed. Repeat PSG at 1 month shows improvement of AHI to 12 without any evidence of side effects. The patient undergoes a repeat treatment of aerosolized CnT and repeat PSG after 1 month shows a normal AHI.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

What is claimed:

1) A method of treating sleep breathing disorders comprising administering a therapeutically effective amount of clostridia neurotoxin (CnT) or light chain thereof to a mammal in need thereof.

2) The method of claim 1, wherein the CnT or light chain thereof is administered to the mammal's nose, nasal cavity, sinuses, oral cavity, pharynx, larynx, trachea or bronchi.

3) The method of claim 1, wherein the sleep breathing disorder is selected from the group consisting of upper airway resistance syndrome, hypopnea, apnea, central sleep apnea, and obstructive sleep apnea.

4) The method of claim 1 wherein the CnT or light chain thereof is administered at a dosage of between about 0.01 to 10,000 units.

5) The method of claim 1 wherein the CnT or light chain thereof is administered at a dosage of between about 0.1 to 1,000 units.

6) The method of claim 1 wherein the CnT or light chain thereof is administered at a dosage of between about 1 to 100 units.

7) The methods of claim 1 wherein the CnT or light chain thereof comprises at least one of the botulinum toxin serotypes A, B, C, D, E, F or G.

8) The method of claim 1 wherein the CnT or light chain thereof is modified by additions or substitutions of at least 1 amino acid.

9) The method of claim 1 wherein the CnT or light chain thereof comprises tetanus toxin.

10) The method claim 1 wherein the CnT or light chain thereof is administered topically.

11) The method of claim 1 wherein the CnT or light chain thereof is administered by injection.

12) The method of claim 11, wherein the injection is pressure jet injection or needle injection.

13) The method of claim 1, wherein the CnT or light chain thereof is administered by aerosolized spray.

14) The method of claim 1 wherein the CnT or light chain thereof is administered using a sustained release delivery method.

15) The method of claim 11, wherein the sustained release delivery method comprises injecting the mammal with a depot injection, administering the CnT or light chain thereof topically, or administering the CnT or light chain thereof in a bioresorbable carrier.

16) The method of claim 1 wherein the CnT or light chain thereof is applied to respiratory or oral mucosa.

17) The method of claim 1 wherein the CnT or light chain thereof is administered across respiratory or oral mucosa.

18) The method of claim 1, wherein the CnT or light chain thereof is administered to the sphenopalatine ganglia.

19) The method of claim 1, wherein the light chain of CnT is administered to the mammal.

20) A method of treating the symptoms of sleep breathing disorders comprising administering a therapeutically effective amount of clostridia neurotoxin (CnT) or light chain thereof to a mammal in need thereof.

21) The method of claim 21, wherein the symptom is snoring.
